(19) United States
(12) Patent Application Publication
(10) Pub. No.: US 2015/0126909 A1
(43) Pub. Date: May 7, 2015

(54) Device for evaluating the distal motor skills of the upper limbs of a person

(71) Applicant: Association Institut de Myologie, Paris (FR)

(72) Inventors: Jean-Yves Hogrel, Montrouge (FR); Laurent Servais, Paris (FR); Aurélie Canal, Paris (FR); Thomas Voit, Boullay-les-Troux (FR)

(21) Appl. No.: 14/380,156

(22) PCT Filed: Feb. 21, 2013
(86) PCT No.: PCT/FR2013/050353

§ 371 Date: Aug. 21, 2014

(30) Foreign Application Priority Data
Feb. 21, 2012 (FR) 1251540

(51) Int. Cl. A61B 5/11; A61B 5/00

(52) U.S. Cl. CPC A61B 5/1125; A61B 5/0004; A61B 5/72; A61B 5/7445; A61B 5/70
USPC 600/595

(57) ABSTRACT

The invention relates to a device for evaluating the distal motor skills of the upper limbs of a person, which includes a main bearing surface (1) for all or part of an upper limb, a measurement means including two push members (2, 3), each of which is provided with a bearing surface (20, 30) lying in a plane parallel to and different from that of said main surface (1), and each of which is coupled to a force sensor capable of generating a signal relating to the force exerted by the individual on said bearing surface, and a control system coupled to the measurement means, said control system making it possible to count a number of alternating pushes on each bearing surface. According to the invention, the push members (or keys) (2, 3) are screwed onto the force sensor, and each force sensor has a precision of around 0.01%.

### TECHNICAL FIELD OF THE INVENTION

The invention relates to the field of evaluating the motor skills of the upper limbs of a person.

More particularly it entails detecting, measuring, memorising and processing measurements relative to the force and to the muscular function of a person in particular to the motor skills of the flexion/extension functions of the wrist and of the fingers of a person.

Evaluating the muscular capacity is particularly interesting in the case of the diagnostic of pathologies affecting the muscles such as myopathies, neurological, rheumatic, traumatic pathologies, sarcopenia linked to age. This evaluation advantageously makes it possible to evaluate the therapeutic effects of a treatment or of an intervention; the measurements of the motor skills indicated hereinabove are the first and main link for such evaluations.

### PRIOR ART

In document FR 2 961 086 a device for evaluating the motor skills of the upper limbs of a person is disclosed, that comprises a shelf provided with a groove, a module for measuring that is mobile in translation along the groove, two push buttons (keys) with each one having a bearing surface of a different and variable height in relation to the surface of the shelf and each one being coupled to a force sensor able to generate a signal correlated to the force of the person who presses the button; a control system is moreover coupled to the module for measuring and it comprises a counter of the actions carried out by the person on each one of the keys as well as a built-in and programmable chronometer that allows for a countdown.

This device fulfils the basic function that is assigned to it, namely detecting and counting the marked pushes of one or of several fingers of a person on each one of the keys that extend beyond the surface of the shelf, in a given amount of time. In other words this device makes it possible to evaluate the flexion/extension skills of the wrist and of the fingers of the hand of a person, as well as to measure the speed of these movements over time.

However this device has a lack of sensitivity in detection, in particular when persons afflicted with neuromuscular disorders are concerned. In these cases the device cannot detect pushes that are too weak, carried out by the patient of which the muscular strength can be too weak to be detected by this device.

Another disadvantage of this known device resides in the contact between each key and the associated sensor placed immediately below. This contact is not reliable in that each key rests via gravity on a sensor. In the event of any vibrations, impacts or movements whatsoever of the device, the contact is disturbed which distorts the measurements. In addition, substantial friction induces wear and tear of the parts in contact which contributes to disrupting the settings of the detection thresholds.

In terms of prior art article entitled “A device for testing the intrinsic muscles of the hand” (Journal of Hand therapy, October 2007, pages 345-350) is also known which discloses a device intended to evaluate the skills of the fingers to move away from one another. This device comprises in particular notches for embedding each one of the fingers, with each notchable to slide along a groove provided for this purpose. The objective of this device is therefore different from that of this invention as well as the means implemented.

### DESCRIPTION OF THE INVENTION

The invention aims to overcome the disadvantages of prior art and in particular propose a device of the type mentioned hereinabove that allows in particular for greater precision as well as greater reliability in the measurements.

To do this, a device for evaluating the distal motor skills of the upper limbs of a person is proposed comprising a main bearing surface for all or a portion of an upper limb, a measurement means comprising two push members each one provided with a bearing surface lying in a plane parallel to and different from that of said main surface and each one coupled to a force sensor able to generate a signal relating to the force exerted by the person on said bearing surface, and a control system coupled to the measurement means, said control system making it possible to count a number of alternating pushes on each bearing surface.

According to a first aspect of the invention said push members are screwed onto the force sensor, occupy a fixed position on the main bearing surface, are intended to be aligned substantially according to the longitudinal direction of the limb in place unsaid surface and each force sensor has a precision of around 0.01%.

The friction, the play and all of the other relative movements are as such avoided between each key and the associated sensor. The reliability and the precision of the measurements are greatly improved. Advantageously each force sensor has an adjustable detection threshold of which the minimum value is of a magnitude of 10 g. This choice advantageously makes it possible to process measurements relating to very weak persons.

Interestingly the reliability of the measurements is improved substantially because the keys are practically no longer sensitive to the vibrations and/or other jerky and undesired movements of the device or of the support whereon it is positioned.

Advantageously the control system comprises a means for adjusting at least one sensitivity threshold of the sensors. As such the user can easily adapt the measurements to each case considered. Preferably three thresholds are chosen.

Preferentially the means for adjusting of at least one threshold is integrated into the device.

Furthermore the device comprises means for transmitting measurements to at least one external means of storage and/or processing of said measurements. These means for transmitting can be wired or wireless.

According to a preferred embodiment of the invention, said main bearing surface is part of a flat support constituted of at least one first element and is able to integrate the measurement means and/or the control system.

Furthermore the flat support can include a second element that can be juxtaposed to the first element in such a way as to increase said main bearing surface. This particularity makes it possible to adapt the size of the device to that of the limb (forearm, wrist, hand) of the person.

Interestingly the flat support is constituted of a material that has a low density such as a polyethylene foam. Reducing the weight of the device facilitates transporting it and its handling.

In addition the material chosen allows for varied and diverse shapes which combine the expected technicity with the overall aesthetics of the device.

The device further comprises a means for displaying the number of actions registered and/or the time elapsed or remaining since the first sequence of actions. Preferentially the means for displaying is incorporated into the support; it can for example be flush with the main surface of the support in order to constitute a functional and aesthetic unit.

Advantageously, said bearing surfaces of the push members are located at altitudes that are equal or different from one another. Setting the altitude is easy and very advantageous.

### BRIEF DESCRIPTION OF THE FIGURES

Other characteristics, details and advantages of the invention shall appear when reading the following description, in reference to the annexed figures, which show:

FIG. 1, a perspective view of a device according to a first embodiment of the invention;

FIG. 2, another perspective view of a device according to a first embodiment of the invention;

FIG. 3, a partial top view of a device according to the invention; and

FIG. 4 a side view of a device according to the invention.

For increased clarity, identical or similar elements are marked with identical reference signs over all of the figures.

### DETAILED DESCRIPTION OF AN EMBODIMENT

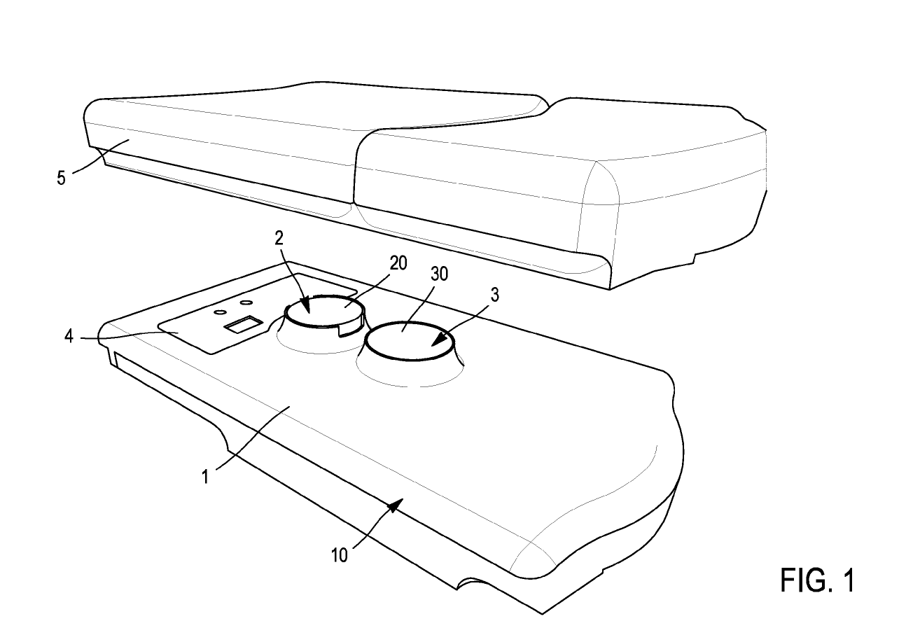

According to the view in FIG. 1 the device for evaluating according to the invention comprises a main bearing surface 1 which is hereof a generally rectangular shape and is used as a bearing for all or a portion of an upper limb of a person, namely his forearm and his wrist. Starting at and above this main bearing surface 1, a first 2 and a second 3 push members (or keys) are provided and extend beyond said bearing surface 1. Each member therefore has a bearing surface 20, 30 lying in a plane parallel to and different from that of the main bearing surface 1. Moreover each push member is coupled with a force sensor able to generate a signal that represents the force exerted by the person on the bearing surface 20, 30 of each push member 2, 3. Moreover a control system is provided, coupled in particular to the force sensors, in order to collect, format and process the signals coming from the sensors.

Interestingly the sensors were chosen in such a way as to have a very low sensitivity, of a magnitude of 10 g or 30 g or 50 g for example. It is as such possible not only to detect very low forces but also an adjustment can be made in order to adapt the sensitivity of the measurements to the subject tested. Relating to the keys 2, 3 themselves, they preferentially have a bearing surface 20, 30 of circular shape; advantageously they are screwed onto each force sensor placed immediately below. A fastening that is of course movable but rigid of the keys 2, 3 on the sensors is favoured so that the efforts on the keys 2,3 can be faithfully reported on each sensor. This therefore makes it possible to improve the reliability of the measurements.

Moreover a precision of around 0.01% characterises each sensor, in order to have a very precise report of each effort on the sensors. For the purposes of illustration the sensors chosen are weighing sensors of the SCAIME registered trademark, reference AQ10.

The push members 2,3 are as such placed in a fixed and given position of the main surface 1 and they are aligned according to a length of said main surface 1.

It is important that they be aligned substantially according to the longitudinal direction of the limb (forearm) placed on said main surface 1. If needed, visual marks can be provided on the main surface in order to show the adequate positioning of the forearm; means such as straps can also be provided not only to position but also to maintain the forearm.

The push members are more preferably of circular shape and they are surrounded on their periphery by a sort of circular frame that rises from the main surface 1. The frame is advantageously moulded with the main surface 1 and a rounded and non-brittle junction surface is provided so that the fingers of the user cannot strike a surface that has a sharp edge.

The bearing surface 20, 30 of the push members 2,3 can advantageously be arranged at an altitude that is different from the main bearing surface 1. Moreover the push members (or keys) 2, 3 can extend beyond the bearing surface 1 differently. Preferably the distal key 2 (the farthest from the patient) is higher than the proximal key 3. This makes it possible to have the patient execute arising movement of the hand and of the wrist, which solicits his extensors of the fingers and of the wrist. For the purposes of illustration the proximal key 3 is placed at 1.5 cm from the main surface 1 while the distal key 2 can be placed at 2.5 cm or at 3.5 cm from said main surface 1. Of course the distal key 2 can be placed at 1.5 cm from the main surface 1. Other altitude values can be provided and chosen in order to increase or on the contrary simplify the muscular work load of the patient. The screwing used in the framework of the invention, between the keys and the associated sensor, makes it possible to easily and precisely adjust the altitude of at least one of the keys 2,3.

Moreover an associated software makes it possible to ‘manage’ the push sequences on the keys: it is thus sought to check that the user pushes alternatively on the keys 2, 3. Otherwise, the software stops the counting i.e. the process of recording measurements. The control system, not shown, comprises a means for adjusting at least one sensitivity threshold of the sensors. This feature makes it possible to adapt the device to the person who must be evaluated. Preferably this means of adjusting is integrated into the body of the device. A lever, control button or sensitive key allows the user to display the sensitivity threshold chosen. Preferably three sensitivity thresholds are available, namely ‘high’, ‘medium’, ‘low’. The user chooses from among one of these, at his convenience and according to the case considered.

The measurements relate for example the number of pushes carried out by the user successively on each one of the keys 2,3, in a given lapse of time. This here involves on the one hand counting and recording this number of pushes anon the other hand associating a chronometer with this operation. This test is known in itself; this invention constitutes a notable improvement in these measurements in particular concerning the reliability and the sensitivity.

As can be seen in the figures, the main bearing surface 1 is part of and/or is fastened onto a flat support constituted of at least one first element 10 able to house the measurement means and/or the control system. The first element 10 is for example made from a polyethylene foam of which the density is low. Materials with a polyamide, polypropylene base can be used without leaving the scope of the invention. For the purposes of illustration a Plastazote foam with a density equal to 45 kg/m3 was chosen with success.

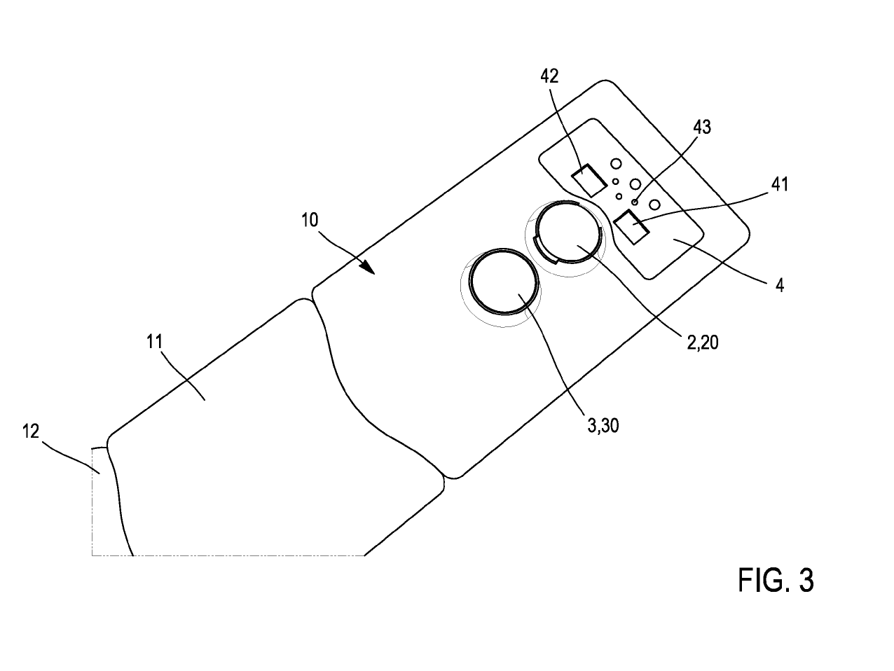

The first element 10 of the support can have diverse and modifiable shapes. For reasons of encumbrance and/or ease of transport, a second element 11 can be provided, that can be juxtaposed to the first element 10. FIG. 3 shows a possible juxtaposition, which makes it possible to extend the main bearing surface 1 and to adapt it to the morphology of the person (adult, child). Possibly a third element 12 can be provided, arranged in the extension of the first two. Those skilled in the art will choose according to the dimensional constraints imposed by the morphology of the patient evaluated.

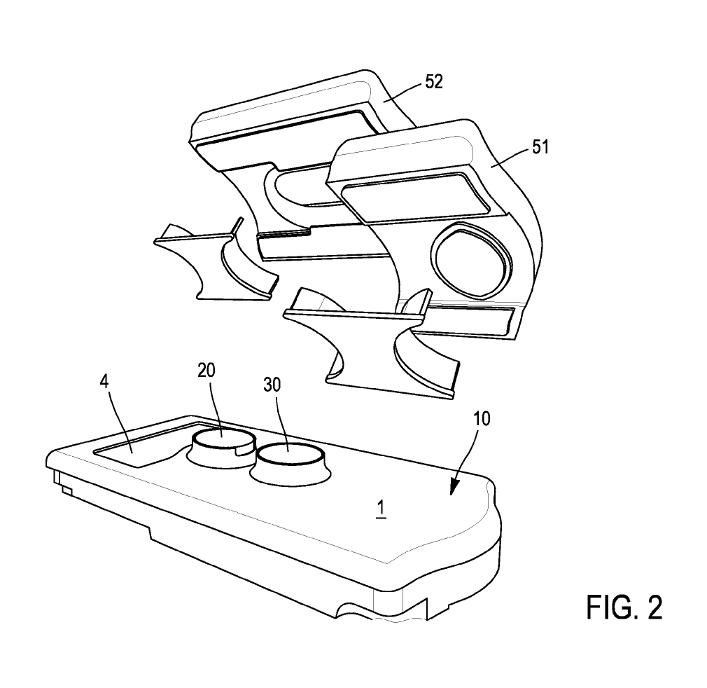

A cover 5; 51,52 can cover and protect the main bearing surface 1 provided with keys 2,3. Any shape, material can be considered for the cover which can be a single piece 5 (FIG. 1) or constituted of several portions 51, 52 (FIG. 2). A transport case can as such be designed, constituted for one portion of the support 10 and for one portion of a cover 5; 51,52. Furthermore the shape of the cover 5 can be as such used as a second bearing element 11 during measurements. Likewise with the third bearing element 12 which can constitute the second portion 52 of the cover 5. The cover 5 and/or each constituent portion 51, 52 therefore has a dual function: protection during transport and/or storage of the device; extending the first element 10 during measurements.

Moreover a means for displaying 4, preferentially integrated into the first element 10 of the support, is provided. It can comprise a module for displaying 41 the number of pushes (or actions), a module for displaying 42 the time elapsed since a given instant or remaining since the last sequence of actions; lighted indicators with each one having a given functionality (sensitivity threshold, power, etc.) can be part of the display; one or several lighted indicators 43 with each one corresponding for example a threshold or a level of sensitivity of the keys 2,3. These displays will be chosen by those skilled in the art in order to adapt the device according to the invention as needed. As can be seen in particular in FIGS. 1,2 and 3 the module for displaying 4 has a transparent surface lying in the same plane as the main bearing surface 1, as such entirely integrated into the flat support 10.

Thanks to a specific preferentially built-in software, it is possible to adjust a finite number of sensitivity thresholds; this therefore makes it possible to adapt the measurements to the motor skills of the person that one desires to evaluate.

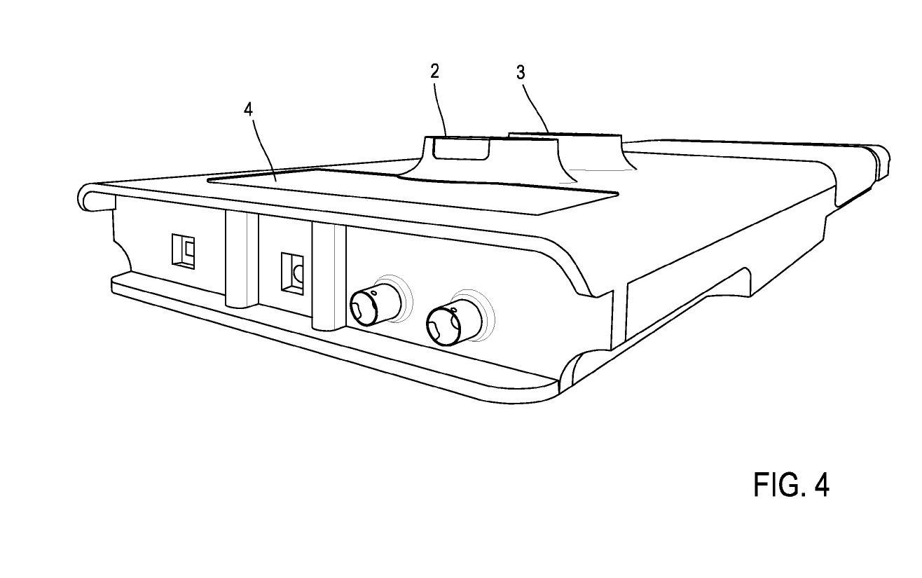

Means for connecting and for transmitting measurements are provided, which can be seen in FIG. 4. The connections can be wired of the RS232 type for example, or be wireless. They make it possible to connect the device according to the invention to means for calculating, viewing, maintaining, etc. or other. A USB port can be provided on the front face of the device. It can be used for example to update the built-in software.

1. Device for evaluating the distal motor skills of the upper limbs of a person comprising a main bearing surface (1) for all or a portion of an upper limb, a measurement means comprising two push members (2, 3) each one provided with a bearing surface (20, 30) lying in a plane parallel to and different from that of said main surface (1) and each one coupled to a force sensor able to generate a signal relating to the force exerted by the person on said bearing surface, and a control system coupled to the measurement means, said control system making it possible to count a number of alternating pushes on each bearing surface (20, 30) characterised in that said push members (2, 3) are screwed onto the force sensor, occupy a fixed position on the main bearing surface (1), are intended to be aligned substantially according to the longitudinal direction of the limb in place on said surface and in that each force sensor has a precision of around 0.01%.

2. Device for evaluating the distal motor skills of the upper limbs of a person according to claim 1 characterised in that each force sensor has an adjustable detection threshold of which the minimum value is of a magnitude of 10 g.

3. Device for evaluating the distal motor skills of the upper limbs of a person according to claim 1 or 2 characterised in that the control system comprises a means for adjusting at least one sensitivity threshold of the sensors.

4. Device for evaluating the distal motor skills of the upper limbs of a person according to claim 3 characterised in that said means for adjusting is integrated into the device.

5. Device for evaluating the distal motor skills of the upper limbs of a person according to claim 1 or 2 characterised in that it comprises means for transmitting measurements to at least one external means for storing and/or for processing said measurements.

6. Device for evaluating the distal motor skills of the upper limbs of a person according to claim 1 or 2 characterised in that said main bearing surface (1) is part of a flat support constituted of at least one first element (10) and able to integrate the measurement means and/or the control system.

7. Device for evaluating the distal motor skills of the upper limbs of a person according to claim 6 characterised in that the flat support comprises a second element (20) that can be juxtaposed to the first element (10) in such a way as to increase said main bearing surface.

8. Device for evaluating the distal motor skills of the upper limbs of a person according to claim 6 characterised in that the flat support (10, 20) is constituted of a material that has a low density such as a polyethylene foam.

9. Device for evaluating the distal motor skills of the upper limbs of a person according to claim 1 or 2 characterised in that it further comprises a means for displaying (4) the number of actions recorded and/or the time elapsed or remaining since the first sequence of actions.

10. Device for evaluating the distal motor skills of the upper limbs of a person according to claim 1 or 2 characterised in that said means for displaying (4) is integrated into said support (10, 20).

11. Device for evaluating the distal motor skills of the upper limbs of a person according to claim 1 or 2 characterised in that said bearing surfaces (20, 30) of the push members (2, 3) are located at altitudes that are equal or different from one another.
